(19) United States
(12) Patent Application Publication
(10) Pub. No.: US 2008/0161856 A1
(43) Pub. Date: Jul. 3, 2008

(54) SPINAL STABILIZATION SYSTEM

(76) Inventors: Mingyan Liu, Bourg la reine (FR); Loic Josse, Denens (CH)

Correspondence Address: LARSON NEWMAN ABEL POLANSKY & WHITE, LLP, 5914 WEST COURTYARD DRIVE, SUITE 200, AUSTIN, TX, 78730, US

(21) Appl. No.: 11/539,407

(22) Filed: Oct. 6, 2006

(51) Int. Cl. A61B 17/70

(52) U.S. Cl. 606/248

(57) ABSTRACT

An anchorage component that can be installed within a spinal stabilization system is disclosed. The anchorage component can include a first lateral half formed with a first spinous process engagement window and a second lateral half formed with a second spinous process engagement window. The first lateral half and the second lateral half can be installed around a spinous process of a vertebra.

### FIELD OF THE DISCLOSURE

The present disclosure relates generally to orthopedics and orthopedic surgery. More specifically, the present disclosure relates to spinal stabilization systems.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a posterior view of a spinal stabilization system;

FIG. 5 is a posterior view of a first embodiment of an anchorage component associated with the spinal stabilization system;

FIG. 6 is an anterior view of the first anchorage component;

FIG. 7 is a posterior view of the spinal stabilization system installed along a spinal column;

FIG. 8 is a posterior view of a second embodiment of an anchorage component associated with the spinal stabilization system;

FIG. 9 is an anterior view of the second anchorage component;

FIG. 10 is a posterior view of a third embodiment of an anchorage component associated with the spinal stabilization system;

FIG. 11 is an anterior view of the third anchorage component; and

FIG. 12 is a flow chart illustrating a method of installing a spinal stabilization system.

### DETAILED DESCRIPTION OF THE DRAWINGS

An anchorage component that can be installed within a spinal stabilization system is disclosed. The anchorage component can include a first lateral half formed with a first spinous process engagement window and a second lateral half formed with a second spinous process engagement window. The first lateral half and the second lateral half can be installed around a spinous process of a vertebra.

In another embodiment, a spinal stabilization system is disclosed and can include a first anchorage component. The first anchorage component can include a first lateral half and second lateral half. Further, the first lateral half and the second lateral half of the first anchorage component can be fitted around a spinous process. The spinal stabilization system can also include a second anchorage component. The second anchorage component can include a first lateral half and second lateral half. The first lateral half and the second lateral half of the second anchorage component can be fitted around a spinous process. The spinal stabilization system can also include a first longitudinal member that can be installed at least partially within the first anchorage component and the second anchorage component.

In still another embodiment, a method of installing a spinal stabilization system is disclosed and can include exposing a portion of a spinal column and installing a first anchorage component around a first spinous process of the spinal column. The first anchorage component can circumscribe the first spinous process.

In yet another embodiment, a kit is disclosed and can include a plurality of anchorage components. Each anchorage component can include a first lateral half and a second lateral half that can be fitted around a spinous process. The kit can also include a plurality of longitudinal members that can be installed within each of the plurality of anchorage components. Also, the kit can include a plurality of setscrews that can bind the longitudinal members within each of the plurality of anchorage components.

### Description of Relevant Anatomy

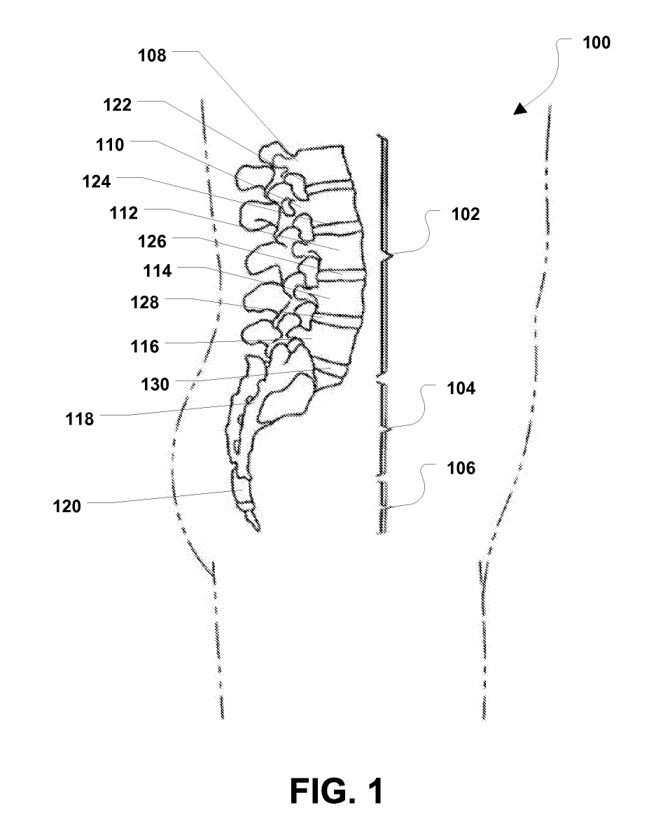

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of repair, treatment of that intervertebral lumbar disc 122, 124, 126, 128, 130 can be effected in accordance with one or more of the embodiments described herein.

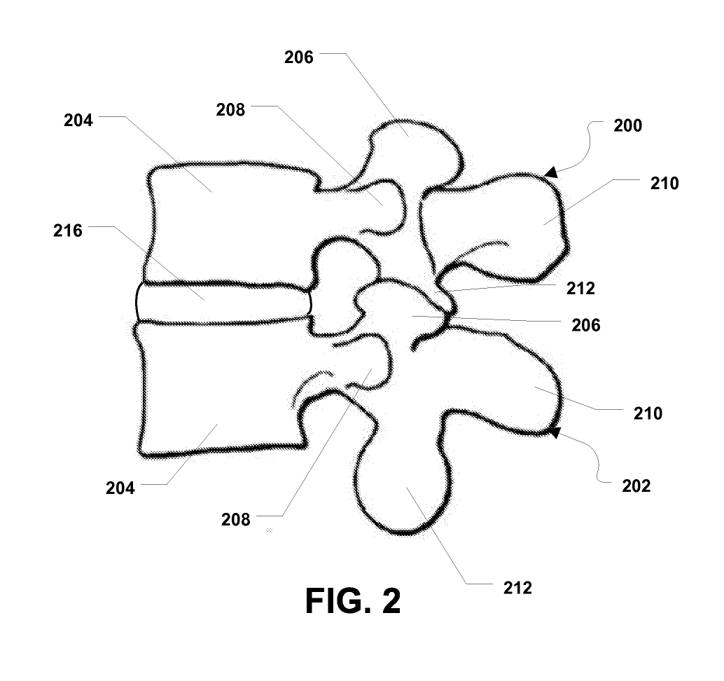

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral disc 216 between the superior vertebra 200 and the inferior vertebra 202.

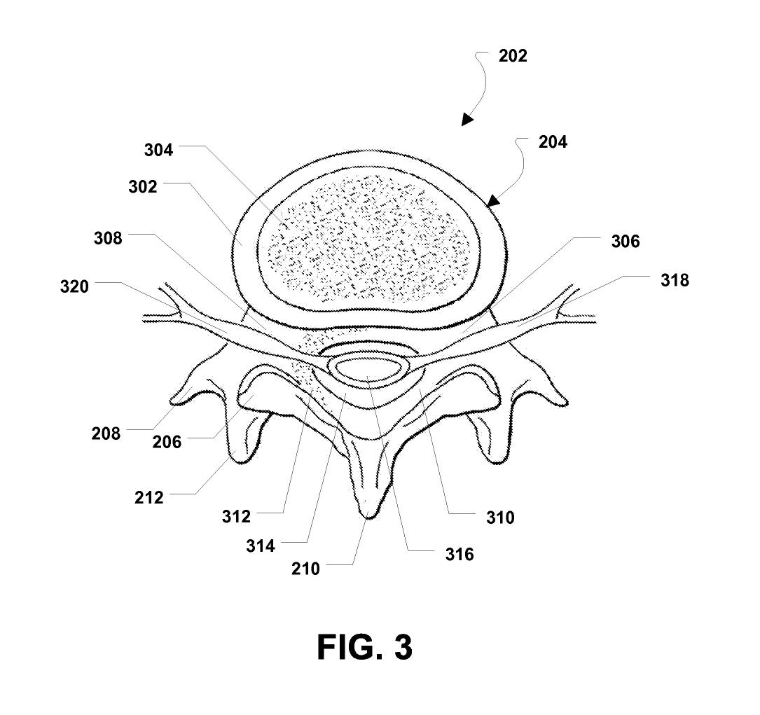

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

### Description of a Spinal Stabilization System

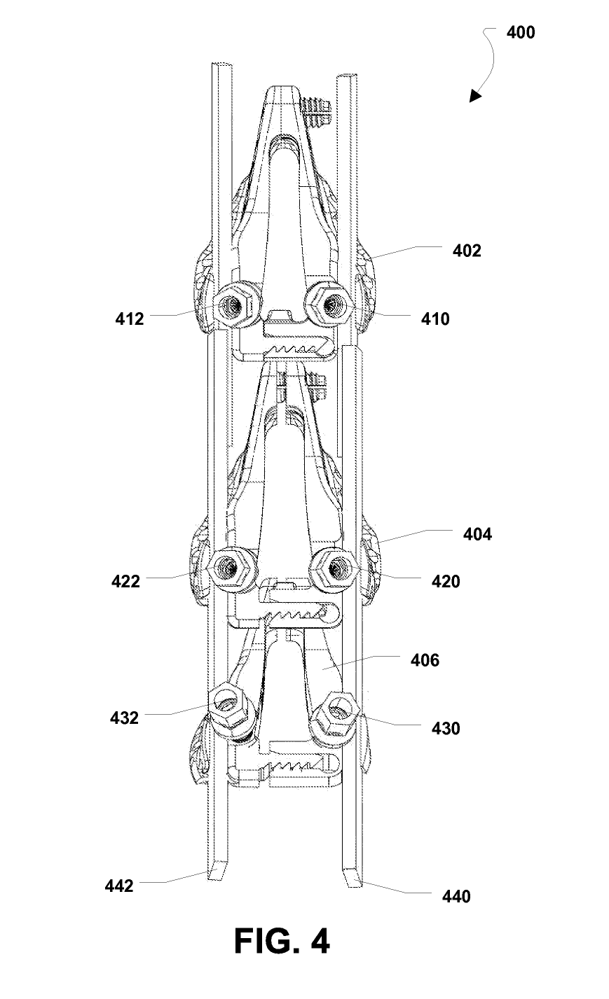

Referring to FIG. 4, a spinal stabilization system is shown and is generally designated 400. As illustrated, the spinal stabilization system 400 can include a first anchorage component 402, a second anchorage component 404, and a third anchorage component 406. In one or more alternative embodiments, the spinal stabilization system 400 can include more than three anchorage components or less than three anchorage components.

In a particular embodiment, the anchorage components 402, 404, 406 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The (PAEK) materials can include polyetherketone (PEK), polyetheretherketone (PEEK), polyetherketoneketone (PEKK), polyetherketoneetherketoneketone (PEKEKK), or a combination thereof. Alternatively, the anchorage components 402, 404, 406 can be made from any other substantially rigid biocompatible materials.

As illustrated in FIG. 4, the first anchorage component 402 can include a first setscrew 410 and a second setscrew 412. The second anchorage component 404 can include a first setscrew 420 and a second setscrew 422. Moreover, the third anchorage component 406 can include a first setscrew 430 and a second setscrew 432. Each setscrew 410, 412, 420, 422, 430, 432 can include a break-off head that can be sheared by a break-off tool at a predetermined torque. As such, each setscrew 410, 412, 420, 422, 430, 432 may not be over-torqued.

FIG. 4 indicates that a first longitudinal element 440 can extend at least partially through each anchorage component 402, 404, 406. In particular, the first longitudinal element 440 can extend through a first slot formed in each anchorage component 402, 404, 406. Further, the first longitudinal element 440 can be held in placed by each first setscrew 410, 412, 422 that extends from each anchorage component 402, 404, 406.

A second longitudinal element 442 can extend at least partially through each anchorage component 402, 404, 406. In particular, the second longitudinal element 442 can extend through a second slot formed in each anchorage component 402, 404, 406. Additionally, the second longitudinal element 442 can be held in placed by each second setscrew 412, 422, 432 that extends from each anchorage component 402, 404, 406. As shown, each longitudinal element 440, 442 can be a bar having a rectangular cross-section. Alternatively, each longitudinal element 440, 442 can have a cross-section that is square, round, elliptical, Y-shaped, U-shaped, any polygonal shape, or a combination thereof.

### Description of a First Embodiment of an Anchorage Component

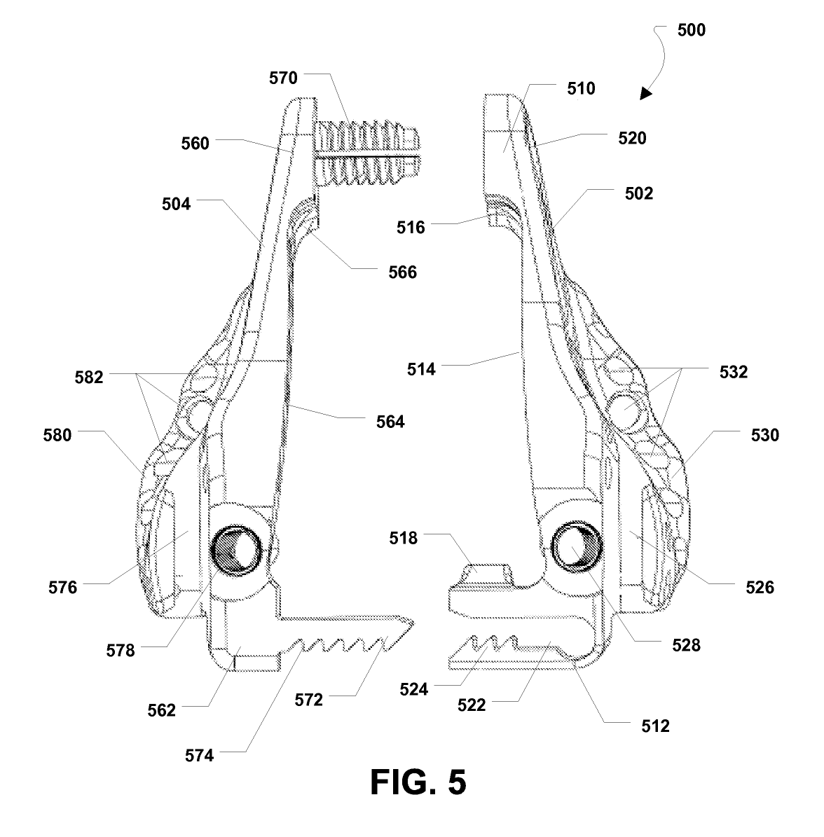

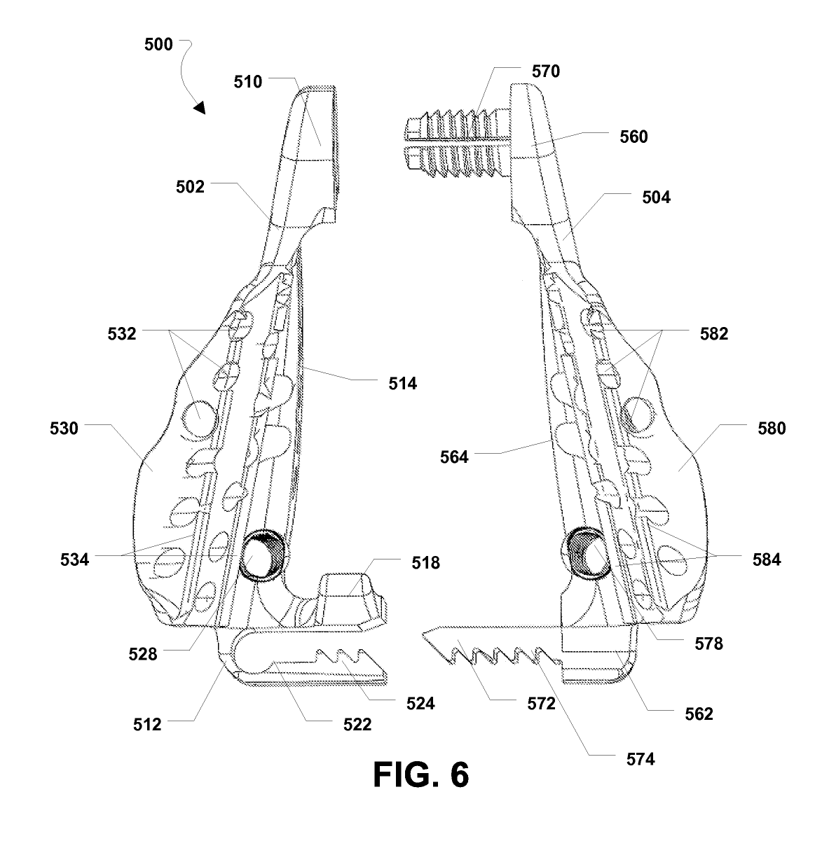

Referring to FIG. 5 and FIG. 6, a first embodiment of an anchorage component is shown and is designated 500. In a particular embodiment, the anchorage component 500 illustrated in FIG. 5 and FIG. 6 can be used in conjunction with the spinal stabilization system 400, described above.

As depicted in FIG. 5 and FIG. 6, the anchorage component 500 can include a first lateral half 502 and a second lateral half 504. The first lateral half 502 can include a superior end 510 and an inferior end 512. A first spinous process engagement window 514 can be established within the first lateral half 502 of the anchorage component 500 between the superior end 510 and the inferior end 512. The first spinous process engagement window 514 can be sized and shaped to allow the first lateral half 502 to be installed partially around a spinous process.

FIG. 5 and FIG. 6 show that the first lateral half 502 of the anchorage component 500 can include a first cutting edge 516 that can extend into the first spinous process engagement window 514 from the superior end 510 of the first lateral half 502. When installed around a spinous process, as described in detail below, the first cutting edge 516 can engage the cephalad end of the laminar. The first lateral half 502 of the anchorage component 500 can also include an infra laminar hook 518 that can extend into the first spinous process engagement window 514 from the inferior end 512 of the first lateral half 502. When the anchorage component 500 is installed around a spinous process, as described in detail below, the infra laminar hook can be inserted under the caudal end of the laminar.

As shown in FIG. 5 and FIG. 6, the superior end 510 of the first lateral half 502 can be formed with a threaded hole 520. The threaded hole 520 can be sized and shaped to receive a post, described below, that can extend from a superior end of the second lateral half. As an alternative to threads, the hole 520 can be formed with a plurality of annular rings or grooves. The inferior end 512 of the first lateral half 502 can be formed with a groove 522 and a plurality of teeth 524 can extend into the groove 522. The groove 522 can be sized and shaped to receive a tongue, described below, that can extend from an inferior end of the second lateral half.

FIG. 5 further shows that the first lateral half 502 can be formed with a first slot 526. The first slot 526 can be sized and shaped to receive a longitudinal element, e.g., the bar shaped longitudinal element described above. Alternatively, the first slot 526 can be sized and shaped to receive a rod, a plate, a blade, a cable, another longitudinal device, or a combination thereof. FIG. 5 also shows that a first threaded setscrew hole 528 can be formed adjacent to, or otherwise near, the first slot 526. The first threaded setscrew hole 528 can be sized and shaped to receive a setscrew, e.g., one of the setscrews described above.

As depicted in FIG. 5 and FIG. 6, the first lateral half 502 can include a first spinous process engagement structure 530 that can extend from the first lateral half 502. In a particular embodiment, the first spinous process engagement structure 530 can extend from the first lateral half 502 adjacent to the first spinous process engagement window 514. Further, the first spinous process engagement structure 530 can be curved to approximate the shape of the spinous process. The first spinous process engagement structure 530 can be formed with a plurality of bone engagement holes 532 therethrough.

After the anchorage component 500 is installed within a patient around a spinous process, the bone engagement holes 532 can allow bone to grow into and around the first lateral half 502 of the anchorage component 500. Further, the first lateral half 502 can be coated with a bone-growth promoting substance, e.g., a hydroxyapatite coating formed of calcium phosphate. Additionally, the first lateral half 502 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 6 illustrates that the first spinous process engagement structure 530 can also include a plurality of protrusions 534. The protrusions 534 can extend from an interior surface of the first spinous process engagement structure 530. Further, the protrusions 534 can be ribs, teeth, keels, or a combination thereof. After installation, the protrusions 534 of the first spinous process engagement structure 530 can engage an outer surface of a spinous process and can minimize relative motion between the first lateral half 502 of the anchorage component 500 and the spinous process.

FIG. 5 and FIG. 6 indicate that the second lateral half 504 can include a superior end 560 and an inferior end 562. A spinous process engagement window 564 can be established within the second lateral half 504 of the anchorage component 500 between the superior end 560 and the inferior end 562. The spinous process engagement window 564 can be sized and shaped to allow the second lateral half 504 to be installed partially around a spinous process.

In a particular embodiment, then the first lateral half 502 and the second lateral half 504 of the anchorage component 500 are installed around a spinous process, as described below, the first spinous process engagement window 514 and the second spinous process engagement window 564 form an opening that can circumscribe the spinous process. The spinous process can extend at least partially through the opening formed by the first spinous process engagement window 514 and the second spinous process engagement window 564.

FIG. 5 and FIG. 6 show that the second lateral half 504 of the anchorage component 500 can include a second cutting edge 566 that can extend into the second spinous process engagement window 564 from the superior end 560 of the second lateral half 504. When installed around a spinous process, as described in detail below, the second cutting edge 566 can engage the cephalad end of the laminar. When the first lateral half 502 and the second lateral half 504 of the anchorage component 500 are installed around a spinous process, as described below, the first cutting edge 516 and the second cutting edge 566 can form a contiguous cutting edge that can engage the cephalad end of the laminar.

As shown in FIG. 5 and FIG. 6, a threaded post 570 can extend from the superior end 560 of the second lateral half 504. The threaded post 570 can be sized and shaped to be received within the threaded hole 520 of the first lateral half 502. As an alternative to threads, the post 570 can be formed with a plurality of annular rings or grooves there around. The threaded post 570 and the threaded hole 520 can establish a first, or superior, connection assembly between the first lateral half 502 and the second lateral half 504 of the anchorage component 500.

The inferior end 562 of the second lateral half 504 can be formed with a tongue 572 and a plurality of teeth 574 can extend from the tongue 572. The tongue 572 can be sized and shaped to be received within the groove 522 formed in the inferior end 512 of the first lateral half 502. The teeth 574 on the tongue 572 can engage the teeth 524 within the groove 522 and can prevent relative motion between the inferior end 512 of the first lateral half 502 and the inferior end 562 of the second lateral half 504. The tongue 572 and the groove 522 can establish a second, or inferior, connection assembly between the first lateral half 502 and the second lateral half 504 of the anchorage component 500.

FIG. 5 further shows that the second lateral half 504 can be formed with a second slot 576. The second slot 576 can be sized and shaped to receive a longitudinal element, e.g., the bar shaped longitudinal element described above. Alternatively, the second slot 576 can be sized and shaped to receive a rod, a plate, a blade, a cable, another longitudinal device, or a combination thereof. FIG. 5 also shows that a second threaded setscrew hole 578 can be formed adjacent to, or otherwise near, the second slot 576. The second threaded setscrew hole 578 can be sized and shaped to receive a setscrew, e.g., one of the setscrews described above.

As depicted in FIG. 5 and FIG. 6, the second lateral half 504 can include a second spinous process engagement structure 580 that can extend from the second lateral half 504. In a particular embodiment, the second spinous process engagement structure 580 can extend from the second lateral half 504 adjacent to the second spinous process engagement window 564. Further, the second spinous process engagement structure 580 can be curved to approximate the shape of the spinous process. The second spinous process engagement structure 580 can be formed with a plurality of bone engagement holes 582 therethrough.

After the anchorage component 500 is installed within a patient around a spinous process, the bone engagement holes 582 can allow bone to grow into and around the second lateral half 504 of the anchorage component 500. Further, the second lateral half 504 can be coated with a bone-growth promoting substance, e.g., a hydroxyapatite coating formed of calcium phosphate. Additionally, the second lateral half 504 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 6 illustrates that the second spinous process engagement structure 580 can also include a plurality of protrusions 584. The protrusions 584 can extend from an interior surface of the second spinous process engagement structure 580. Further, the protrusions 584 can be ribs, teeth, keels, or a combination thereof. After installation, the protrusions 584 of the second spinous process engagement structure 580 can engage an outer surface of a spinous process and can minimize relative motion between the second lateral half 504 of the anchorage component 500 and the spinous process.

### Description of an Installation of a Spinal Stabilization System along a Spinal Column

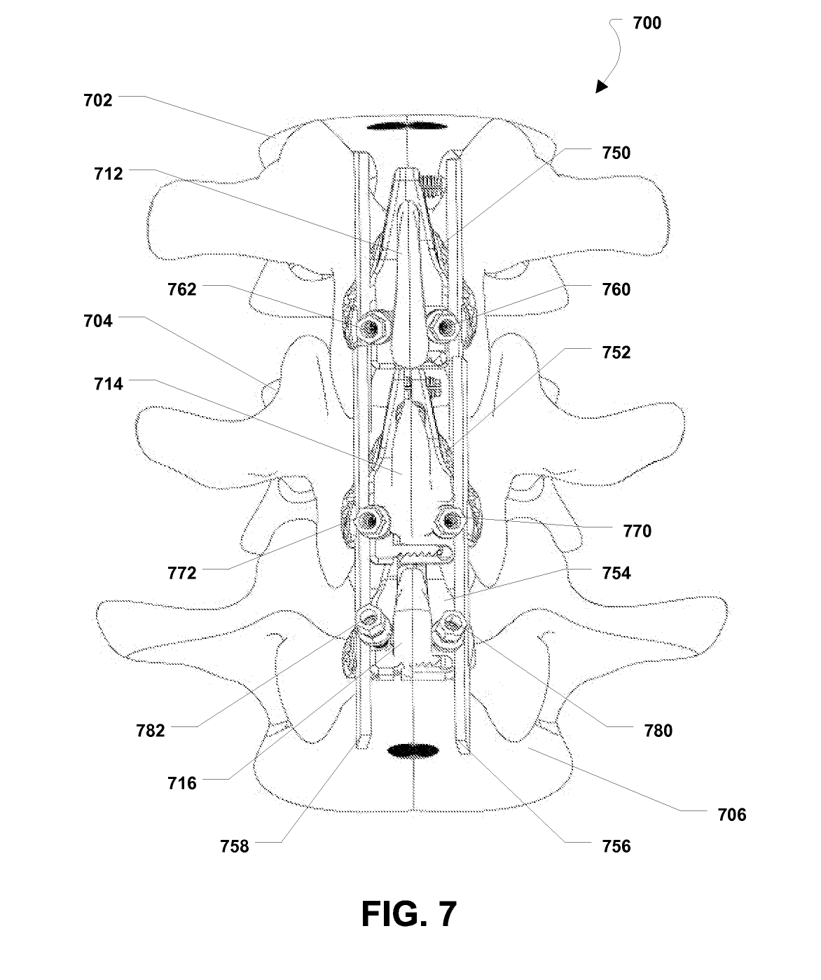

Referring to FIG. 7, a spinal stabilization system is shown installed along a portion of a spinal column, designated 700. The spinal column 700 can include a first vertebra 702, a second vertebra 704, and a third vertebra 706. Moreover, the first vertebra 702 can include a first spinous process 712. The second vertebra 704 can include a second spinous process 714. Also, the third vertebra 706 can include a third spinous process 716.

As shown in FIG. 7, a plurality of anchorage components can be installed along the spinal column 700. In particular, a first anchorage component 750 can be installed around the first spinous process 712, a second anchorage component 752 can be installed around the second spinous process 714, and a third anchorage component 754 can be installed around the third spinous process 716. Each anchorage component 750, 752, 754 can be configured according to the one or more embodiments described herein. Further, each anchorage component 750, 752, 754 can include a first lateral half and a second lateral half and the anchorage components 750, 752, 754 can be installed such that each anchorage component 750, 752, 754 circumscribes a respective spinous process 712, 714, 716.

Further, in a particular embodiment, each anchorage component 750, 752, 754 can include a cutting edge that can engage the cephalad end of the laminar. Also, each anchorage component 750, 752, 754 can include an infra laminar hook that can be inserted under the caudal end of the laminar.

After the anchorage components 750, 752, 754 are installed as shown, a first longitudinal member 756 and a second longitudinal member 758 can be installed along each anchorage component 750, 752, 754. The first anchorage component 750 can include a first setscrew 760 that can hold the first longitudinal member 756 therein. The first anchorage component 750 can also include a second setscrew 762 that can hold the second longitudinal member 758 therein. Additionally, the second anchorage component 752 can include a first setscrew 770 that can hold the first longitudinal member 756 therein. The second anchorage component 752 can also include a second setscrew 772 that can hold the second longitudinal member 758 therein. Further, the third anchorage component 754 can include a first setscrew 780 that can hold the first longitudinal member 756 therein. The third anchorage component 754 can also include a second setscrew 782 that can hold the second longitudinal member 758 therein.

### Description of a Second Embodiment of an Anchorage Component

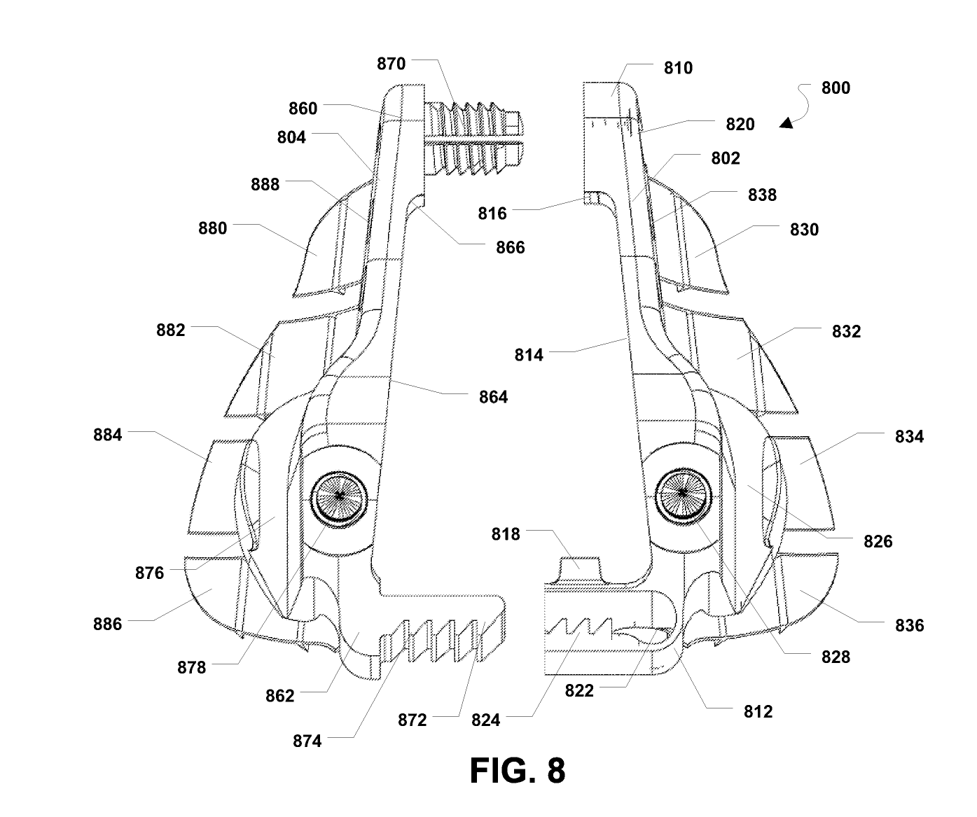

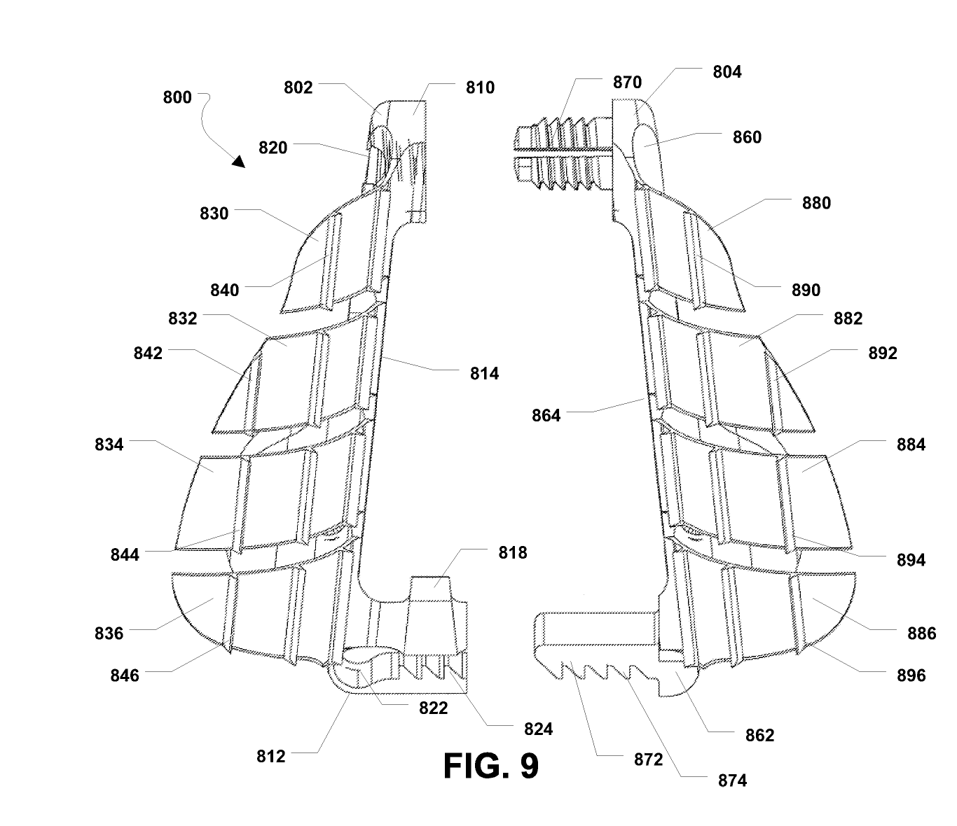

Referring to FIG. 8 and FIG. 9, a second embodiment of an anchorage component is shown and is designated 800. In a particular embodiment, the anchorage component 800 illustrated in FIG. 8 and FIG. 9 can be used in conjunction with the spinal stabilization system 400, described above.

As depicted in FIG. 8 and FIG. 9, the anchorage component 800 can include a first lateral half 802 and a second lateral half 804. The first lateral half 802 can include a superior end 810 and an inferior end 812. A first spinous process engagement window 814 can be established within the first lateral half 802 of the anchorage component 800 between the superior end 810 and the inferior end 812. The first spinous process engagement window 814 can be sized and shaped to allow the first lateral half 802 to be installed partially around a spinous process.

FIG. 8 and FIG. 9 show that the first lateral half 802 of the anchorage component 800 can include a first cutting edge 816 that can extend into the first spinous process engagement window 814 from the superior end 810 of the first lateral half 802. When installed around a spinous process, as described in detail below, the first cutting edge 816 can engage the cephalad end of the laminar. The first lateral half 802 of the anchorage component 800 can also include an infra laminar hook 818 that can extend into the first spinous process engagement window 814 from the inferior end 812 of the first lateral half 802. When the anchorage component 800 is installed around a spinous process, as described in detail below, the infra laminar hook can be inserted under the caudal end of the laminar.

As shown in FIG. 8 and FIG. 9, the superior end 810 of the first lateral half 802 can be formed with a threaded hole 820. The threaded hole 820 can be sized and shaped to receive a post, described below, that can extend from a superior end of the second lateral half. As an alternative to threads, the hole 820 can be formed with a plurality of annular rings or grooves. The inferior end 812 of the first lateral half 802 can be formed with a groove 822 and a plurality of teeth 824 can extend into the groove 822. The groove 822 can be sized and shaped to receive a tongue, described below, that can extend from an inferior end of the second lateral half.

FIG. 8 further shows that the first lateral half 802 can be formed with a first slot 826. The first slot 826 can be sized and shaped to receive a longitudinal element, e.g., the bar shaped longitudinal element described above. Alternatively, the first slot 826 can be sized and shaped to receive a rod, a plate, a blade, a cable, another longitudinal device, or a combination thereof. FIG. 8 also shows that a first threaded setscrew hole 828 can be formed adjacent to, or otherwise near, the first slot 826. The first threaded setscrew hole 828 can be sized and shaped to receive a setscrew, e.g., one of the setscrews described above.

As depicted in FIG. 8 and FIG. 9, the first lateral half 802 can include a first spinous process engagement structure 830, a second spinous engagement structure 832, a third spinous process engagement structure 834, and a fourth spinous process engagement structure 836 that can extend from the first lateral half 802. In a particular embodiment, the spinous process engagement structures 830, 832, 834, 836 can extend from the first lateral half 802 adjacent to the first spinous process engagement window 814. Further, the spinous process engagement structures 830, 832, 834, 836 can be curved to approximate the shape of the spinous process. In a particular embodiment, the spinous process engagement structures 830, 832, 834, 836 can also be at least partially flexible in order to allow the spinous process engagement structures 830, 832, 834, 836 to bend and substantially adapt to the shape of a posterior arch of the vertebra around which the anchorage component 800 is installed. The first lateral half 802 can also be formed with a plurality of bone engagement holes 838 therethrough.

After the anchorage component 800 is installed within a patient around a spinous process, the bone engagement holes 838 can allow bone to grow into and around the first lateral half 802 of the anchorage component 800. Further, the first lateral half 802 can be coated with a bone-growth promoting substance, e.g., a hydroxyapatite coating formed of calcium phosphate. Additionally, the first lateral half 802 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 9 illustrates that the first spinous process engagement structure 830 can also include a plurality of protrusions 840. The protrusions 840 can extend from an interior surface of the first spinous process engagement structure 830. The second spinous process engagement structure 832 can include a plurality of protrusions 842 that can extend from an interior surface of the second spinous process engagement structure 832. The third spinous process engagement structure 834 can include a plurality of protrusions 844 that can extend from an interior surface of the second spinous process engagement structure 834. Also, the fourth spinous process engagement structure 836 can include a plurality of protrusions 846 that can extend from an interior surface of the fourth spinous process engagement structure 836. In a particular embodiment, the protrusions 840, 842, 844, 846 can be ribs, teeth, keels, or a combination thereof.

After installation, the protrusions 840, 842, 844, 846 of the spinous process engagement structures 830, 832, 834, 836 can engage an outer surface of a spinous process and can minimize relative motion between the first lateral half 802 of the anchorage component 800 and the spinous process.

FIG. 8 and FIG. 9 indicate that the second lateral half 804 can include a superior end 860 and an inferior end 862. A spinous process engagement window 864 can be established within the second lateral half 804 of the anchorage component 800 between the superior end 860 and the inferior end 862. The spinous process engagement window 864 can be sized and shaped to allow the second lateral half 804 to be installed partially around a spinous process.

In a particular embodiment, then the first lateral half 802 and the second lateral half 804 of the anchorage component 800 are installed around a spinous process, as described below, the first spinous process engagement window 814 and the second spinous process engagement window 864 form an opening that can circumscribe the spinous process. The spinous process can extend at least partially through the opening formed by the first spinous process engagement window 814 and the second spinous process engagement window 864.

FIG. 8 and FIG. 9 show that the second lateral half 804 of the anchorage component 800 can include a second cutting edge 866 that can extend into the second spinous process engagement window 864 from the superior end 860 of the second lateral half 804. When installed around a spinous process, as described in detail below, the second cutting edge 866 can engage the cephalad end of the laminar. When the first lateral half 802 and the second lateral half 804 of the anchorage component 800 are installed around a spinous process, as described below, the first cutting edge 816 and the second cutting edge 866 can form a contiguous cutting edge that can engage the cephalad end of the laminar.

As shown in FIG. 8 and FIG. 9, a threaded post 870 can extend from the superior end 860 of the second lateral half 804. The threaded post 870 can be sized and shaped to be received within the threaded hole 820 of the first lateral half 802. As an alternative to threads, the post 870 can be formed with a plurality of annular rings or grooves there around. The inferior end 862 of the second lateral half 804 can be formed with a tongue 872 and a plurality of teeth 874 can extend from the tongue 872. The tongue 872 can be sized and shaped to be received within the groove 822 formed in the inferior end 812 of the first lateral half 802. The teeth 874 on the tongue 872 can engage the teeth 824 within the groove 822 and can prevent relative motion between the inferior end 812 of the first lateral half 802 and the inferior end 862 of the second lateral half 804.

FIG. 8 further shows that the second lateral half 804 can be formed with a second slot 876. The second slot 876 can be sized and shaped to receive a longitudinal element, e.g., the bar shaped longitudinal element described above. Alternatively, the second slot 876 can be sized and shaped to receive a rod, a plate, a blade, a cable, another longitudinal device, or a combination thereof. FIG. 8 also shows that a second threaded setscrew hole 878 can be formed adjacent to, or otherwise near, the second slot 876. The second threaded setscrew hole 878 can be sized and shaped to receive a setscrew, e.g., one of the setscrews described above.

As depicted in FIG. 8 and FIG. 9, the second lateral half 804 can include a first spinous process engagement structure 880, a second spinous engagement structure 882, a third spinous process engagement structure 884, and a fourth spinous process engagement structure 886 that can extend from the second lateral half 804. In a particular embodiment, the spinous process engagement structures 880, 882, 884, 886 can extend from the second lateral half 804 adjacent to the second spinous process engagement window 864. Further, the spinous process engagement structures 880, 882, 884, 886 can be curved to approximate the shape of the spinous process. In a particular embodiment, the spinous process engagement structures 880, 882, 884, 886 can also be at least partially flexible in order to allow the spinous process engagement structures 880, 882, 884, 886 to bend and substantially adapt to the shape of a posterior arch of the vertebra around which the anchorage component 800 is installed. The second lateral half 804 can also be formed with a plurality of bone engagement holes 888 therethrough.

After the anchorage component 800 is installed within a patient around a spinous process, the bone engagement holes 888 can allow bone to grow into and around the second lateral half 804 of the anchorage component 800. Further, the second lateral half 804 can be coated with a bone-growth promoting substance, e.g., a hydroxyapatite coating formed of calcium phosphate. Additionally, the second lateral half 804 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 9 illustrates that the first spinous process engagement structure 880 can also include a plurality of protrusions 890. The protrusions 890 can extend from an interior surface of the first spinous process engagement structure 880. The second spinous process engagement structure 882 can include a plurality of protrusions 892 that can extend from an interior surface of the second spinous process engagement structure 882. The third spinous process engagement structure 884 can include a plurality of protrusions 894 that can extend from an interior surface of the second spinous process engagement structure 884. Also, the fourth spinous process engagement structure 886 can include a plurality of protrusions 896 that can extend from an interior surface of the fourth spinous process engagement structure 886. In a particular embodiment, the protrusions 890, 892, 894, 896 can be ribs, teeth, keels, or a combination thereof.

After installation, the protrusions 890, 892, 894, 896 of the spinous process engagement structures 880, 882, 884, 886 can engage an outer surface of a spinous process and can minimize relative motion between the second lateral half 804 of the anchorage component 800 and the spinous process.

### Description of a Third Embodiment of an Anchorage Component

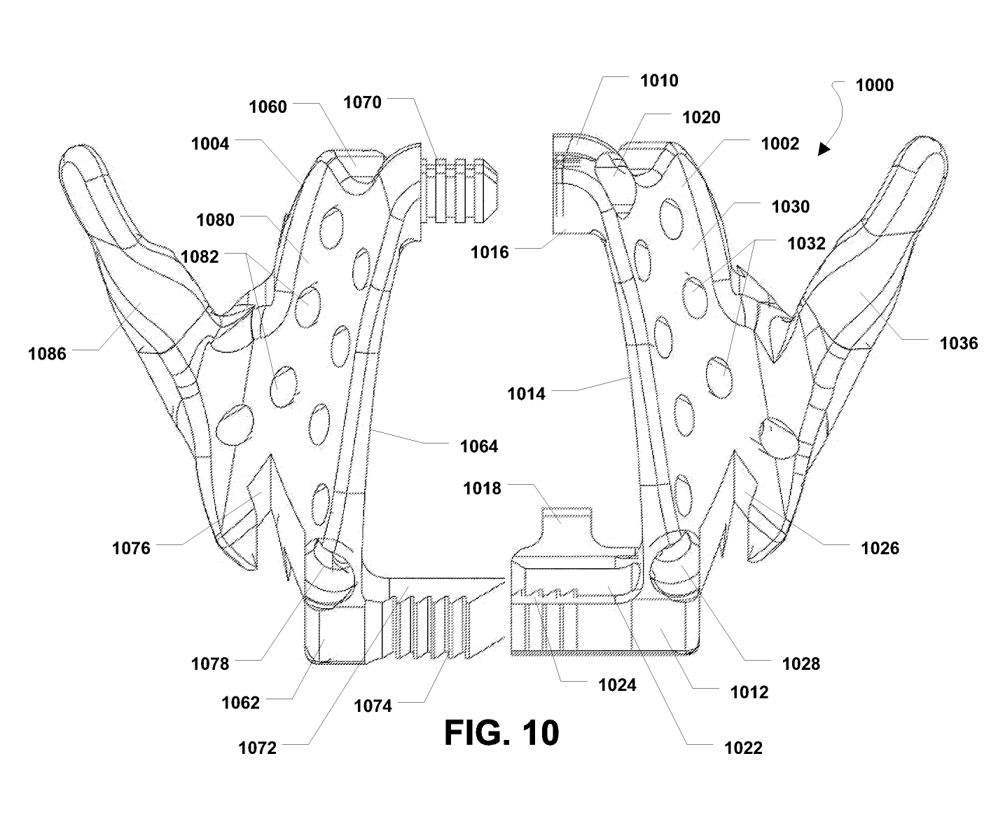

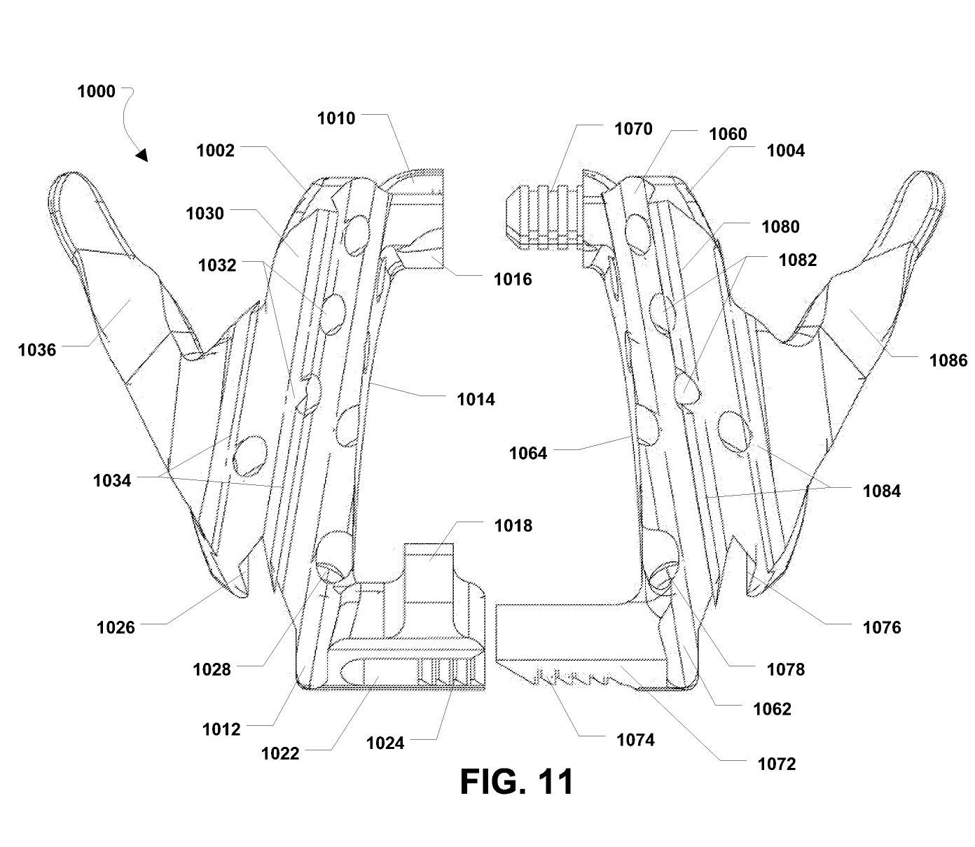

Referring to FIG. 10 and FIG. 11, a third embodiment of an anchorage component is shown and is designated 1000. In a particular embodiment, the anchorage component 1000 illustrated in FIG. 10 and FIG. 11 can be used in conjunction with the spinal stabilization system 400, described above.

As depicted in FIG. 10 and FIG. 11, the anchorage component 1000 can include a first lateral half 1002 and a second lateral half 1004. The first lateral half 1002 can include a superior end 1010 and an inferior end 1012. A first spinous process engagement window 1014 can be established within the first lateral half 1002 of the anchorage component 1000 between the superior end 1010 and the inferior end 1012. The first spinous process engagement window 1014 can be sized and shaped to allow the first lateral half 1002 to be installed partially around a spinous process.

FIG. 10 and FIG. 11 show that the first lateral half 1002 of the anchorage component 1000 can include a cutting edge 1016 that can extend into the first spinous process engagement window 1014 from the superior end 1010 of the first lateral half 1002. When installed around a spinous process, as described in detail below, the cutting edge 1016 can engage the cephalad end of the laminar. The first lateral half 1002 of the anchorage component 1000 can also include an infra laminar hook 1018 that can extend into the first spinous process engagement window 1014 from the inferior end 1012 of the first lateral half 1002. When the anchorage component 1000 is installed around a spinous process, as described in detail below, the infra laminar hook can be inserted under the caudal end of the laminar.

As shown in FIG. 10 and FIG. 11, the superior end 1010 of the first lateral half 1002 can be formed with a threaded hole 1020. The threaded hole 1020 can be sized and shaped to receive a post, described below, that can extend from a superior end of the second lateral half. As an alternative to threads, the hole 1020 can be formed with a plurality of annular rings or grooves. The inferior end 1012 of the first lateral half 1002 can be formed with a groove 1022 and a plurality of teeth 1024 can extend into the groove 1022. The groove 1022 can be sized and shaped to receive a tongue, described below, that can extend from an inferior end of the second lateral half.

FIG. 10 further shows that the first lateral half 1002 can be formed with a first slot 1026. The first slot 1026 can be sized and shaped to receive a longitudinal element, e.g., the bar shaped longitudinal element described above. Alternatively, the first slot 1026 can be sized and shaped to receive a rod, a plate, a blade, a cable, another longitudinal device, or a combination thereof. FIG. 10 also shows that a first threaded setscrew hole 1028 can be formed adjacent to, or otherwise near, the first slot 1026. The first threaded setscrew hole 1028 can be sized and shaped to receive a setscrew, e.g., one of the setscrews described above.

As depicted in FIG. 10 and FIG. 11, the first lateral half 1002 can include a first spinous process engagement structure 1030 that can extend from the first lateral half 1002. In a particular embodiment, the first spinous process engagement structure 1030 can extend from the first lateral half 1002 adjacent to the first spinous process engagement window 1014. Further, the first spinous process engagement structure 1030 can be curved to approximate the shape of the spinous process. The first spinous process engagement structure 1030 can be formed with a plurality of bone engagement holes 1032 therethrough.

After the anchorage component 1000 is installed within a patient around a spinous process, the bone engagement holes 1032 can allow bone to grow into and around the first lateral half 1002 of the anchorage component 1000. Further, the first lateral half 1002 can be coated with a bone-growth promoting substance, e.g., a hydroxyapatite coating formed of calcium phosphate. Additionally, the first lateral half 1002 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 11 illustrates that the first spinous process engagement structure 1030 can also include a plurality of protrusions 1034. The protrusions 1034 can extend from an interior surface of the first spinous process engagement structure 1030. Further, the protrusions 1034 can be ribs, teeth, keels, or a combination thereof. After installation, the protrusions 1034 of the first spinous process engagement structure 1030 can engage an outer surface of a spinous process and can minimize relative motion between the first lateral half 1002 of the anchorage component 1000 and the spinous process.

Further, the first lateral half 1002 can include a first pedicle structure 1036 that can extend from the first spinous process engagement structure 1030 of the first lateral half 1002. The first pedicle structure 1036 can be at least partially flexible to allow the first pedicle structure 1036 to substantially grip an isthmus of the vertebra around which the anchorage component 1000 is installed. The first pedicle structure 1036 can increase the attachment of the first lateral half 1002 of the anchorage component 100 to a spinal process and surrounding bony tissue. Further, the first pedicle structure 1036 can substantially increase the stability of the anchorage component 1000. In a particular embodiment, the first pedicle structure 1036 can be modular and can be installed on the first lateral half 1002 of the anchorage component 1000 at the discretion of the surgeon installing the anchorage component 1000.

FIG. 10 and FIG. 11 indicate that the second lateral half 1004 can include a superior end 1060 and an inferior end 1062. A spinous process engagement window 1064 can be established within the second lateral half 1004 of the anchorage component 1000 between the superior end 1060 and the inferior end 1062. The spinous process engagement window 1064 can be sized and shaped to allow the second lateral half 1004 to be installed partially around a spinous process.

In a particular embodiment, then the first lateral half 1002 and the second lateral half 1004 of the anchorage component 1000 are installed around a spinous process, as described below, the first spinous process engagement window 1014 and the second spinous process engagement window 1064 form an opening that can circumscribe the spinous process. The spinous process can extend at least partially through the opening formed by the first spinous process engagement window 1014 and the second spinous process engagement window 1064.

As shown in FIG. 10 and FIG. 11, a threaded post 1070 can extend from the superior end 1060 of the second lateral half 1004. The threaded post 1070 can be sized and shaped to be received within the threaded hole 1020 of the first lateral half 1002. As an alternative to threads, the post 1070 can be formed with a plurality of annular rings or grooves there around. The inferior end 1062 of the second lateral half 1004 can be formed with a tongue 1072 and a plurality of teeth 1074 can extend from the tongue 1072. The tongue 1072 can be sized and shaped to be received within the groove 1022 formed in the inferior end 1012 of the first lateral half 1002. The teeth 1074 on the tongue 1072 can engage the teeth 1024 within the groove 1022 and can prevent relative motion between the inferior end 1012 of the first lateral half 1002 and the inferior end 1062 of the second lateral half 1004.

FIG. 10 further shows that the second lateral half 1004 can be formed with a second slot 1076. The second slot 1076 can be sized and shaped to receive a longitudinal element, e.g., the bar shaped longitudinal element described above. Alternatively, the second slot 1076 can be sized and shaped to receive a rod, a plate, a blade, a cable, another longitudinal device, or a combination thereof. FIG. 10 also shows that a second threaded setscrew hole 1078 can be formed adjacent to, or otherwise near, the second slot 1076. The second threaded setscrew hole 1078 can be sized and shaped to receive a setscrew, e.g., one of the setscrews described above.

As depicted in FIG. 10 and FIG. 11, the second lateral half 1004 can include a second spinous process engagement structure 1080 that can extend from the second lateral half 1004. In a particular embodiment, the second spinous process engagement structure 1080 can extend from the second lateral half 1004 adjacent to the second spinous process engagement window 1064. Further, the second spinous process engagement structure 1080 can be curved to approximate the shape of the spinous process. The second spinous process engagement structure 1080 can be formed with a plurality of bone engagement holes 1082 therethrough.

After the anchorage component 1000 is installed within a patient around a spinous process, the bone engagement holes 1082 can allow bone to grow into and around the second lateral half 1004 of the anchorage component 1000. Further, the second lateral half 1004 can be coated with a bone-growth promoting substance, e.g., a hydroxyapatite coating formed of calcium phosphate. Additionally, the second lateral half 1004 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 11 illustrates that the second spinous process engagement structure 1080 can also include a plurality of protrusions 1084. The protrusions 1084 can extend from an interior surface of the second spinous process engagement structure 1080. Further, the protrusions 1084 can be ribs, teeth, keels, or a combination thereof. After installation, the protrusions 1084 of the second spinous process engagement structure 1080 can engage an outer surface of a spinous process and can minimize relative motion between the second lateral half 1004 of the anchorage component 1000 and the spinous process.

Further, the second lateral half 1004 can include a second pedicle structure 1086 that can extend from the second spinous process engagement structure 1080 of the second lateral half 1004. The second pedicle structure 1086 can be at least partially flexible to allow the second pedicle structure 1086 to substantially grip an isthmus of the vertebra around which the anchorage component 1000 is installed. The second pedicle structure 1086 can increase the attachment of the second lateral half 1004 of the anchorage component 100 to a spinal process and surrounding bony tissue. Further, the second pedicle structure 1086 can substantially increase the stability of the anchorage component 1000. In a particular embodiment, the second pedicle structure 1086 can be modular and can be installed on the second lateral half 1004 of the anchorage component 1000 at the discretion of the surgeon installing the anchorage component 1000.

### Description of a Method of Installing a Spinal Stabilization System

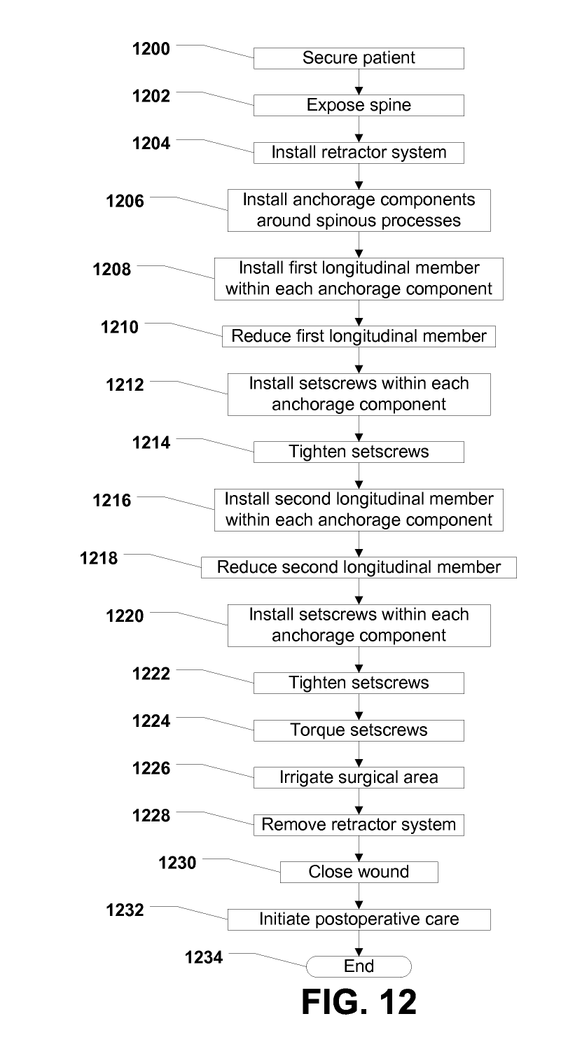

Referring to FIG. 12, an exemplary, non-limiting embodiment of a method of installing a spinal stabilization system is shown and commences at block 1200. At block 1200, a patient is secured on an operating table. For example, the patient can be secured in a prone position to allow a posterior approach to be used to access the patien's spinal column.

Moving to block 1202, the surgical area along spinal column is exposed. Further, at block 1204, a surgical retractor system can be installed to keep the surgical field open. For example, the surgical retractor system can be a surgical retractor system configured for posterior access to a spinal column.

Proceeding to block 1206, the anchorage components of the spinal stabilization system can be installed. For example, a first lateral half of an anchorage component can be laterally installed around a spinous process so that a first spinous process engagement window of the first lateral half at least partially circumscribes the spinous process. Further, an infra laminar hook of the first lateral half can be inserted under a caudal end of the laminar. After the first lateral half is installed, a second lateral half can be laterally installed around the spinous process—from the opposite side of the spinous process relative to the first lateral half. A second spinous process window of the second lateral half of the anchorage component can at least partially circumscribe the spinous process. The second lateral half can be position so that a post that extends from a superior end of the second lateral half can engage a hole formed in a superior end of the first lateral half. Also, a tongue that extends from an inferior end of the second lateral half can engage a groove formed in an inferior end of the first lateral half. Multiple anchorage components, that are similarly configured, can be installed along the spinal column around the spinous processes of adjacent vertebra.

Moving to block 1208, a first longitudinal member can be installed along the anchorage components so that the first longitudinal member is within or near a first slot formed in each anchorage component. At block 1210, the first longitudinal member can be reduced. In other words, a tool, e.g., a reducer, an approximator, an introducer, a persuader, or a combination thereof, can be used to move the longitudinal member into the first slot formed in each anchorage component. At block 1212, setscrews can be installed within each anchorage component, e.g., within a threaded hole adjacent to each first slot. The setscrews can hold the first longitudinal component in place relative to each anchorage component of the spinal stabilization system. At block 1214, each setscrew can be tightened, e.g., using a nut driver or other similar tool.

Continuing to block 1216, a second longitudinal member can be installed along the anchorage components so that the second longitudinal member is within or near a second slot formed in each anchorage component. At block 1218, the second longitudinal member can be reduced as described above. At block 1220, setscrews can be installed within each anchorage component, e.g., within a threaded hole adjacent to each second slot. The setscrews can hold the second longitudinal component in place relative to each anchorage component of the spinal stabilization system. At block 1222, each setscrew can be tightened, e.g., using a nut driver or other similar tool.

At block 1224, each setscrew can be torqued using a break-off tool in order to shear a break-off cap of each setscrew. This can ensure that each setscrew is torqued to approximately the same torque value. At block 1226, the intervertebral space can be irrigated. Further, at block 1228, the retractor system can be removed. At block 1230, the surgical wound can be closed. The surgical wound can be closed using sutures, surgical staples, or any other surgical technique well known in the art. Moving to block 1232, postoperative care can be initiated. The method can end at state 1234.

### Conclusion

With the configuration of structure described above, the spinal stabilization system provides a device that may be implanted to support or stabilize at least a portion of a spinal column that is diseased, degenerated, or otherwise damaged. Further, each anchorage component of the spinal stabilization system can be fitted around a spinous process and one or more longitudinal members can be installed along the anchorage components to provide support and stability for the spinal column.

In one or more of the embodiments described herein, each anchorage component can be configured to attach to, or engage, the laminar surfaces of a vertebra. More specifically, each anchorage component can be configured to engage the junction between the spinous process and the laminar of the vertebra. This laminar spinous part of the vertebra is formed with regularly bi-plane sloping surfaces, i.e., a caudal-to-cephalad sloping surface and a medial-to-lateral sloping surface.

By tightening the connection assemblies of each anchorage component, each half of each anchorage component can be pulled together against these sloping surfaces. Further, the infra laminar hook of each anchorage component can engage the caudal end of the laminar and substantially prevent the anchorage component from moving back along the spinous process. The relatively high strength of the laminar spinous junction posterior to the vertebra and the configuration of the anchorage component can allow the anchorage component to control the vertebra in all directions. As such, spinal fixation using the anchorage components described herein can be very effective.

According to one or more of the embodiments described herein, an anchorage component can include a superior connection assembly and an inferior connection assembly. As described herein, these connection assemblies can be threaded connection assemblies, or tongue-and-groove assemblies. Alternatively, at least one of the connection assemblies can include a hinge and the anchorage component can have a general “clam shell” configuration. In such a case, the anchorage component can be closed around a spinous process until a connection assembly opposite the hinged connection assembly is secured, e.g., by a threaded assembly, a tongue-and-groove assembly, or another securing assembly.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An anchorage component configured to be installed within a spinal stabilization system, the anchorage component comprising:
a first lateral half formed with a first spinous process engagement window; and
a second lateral half formed with a second spinous process engagement window, wherein the first lateral half and the second lateral half are configured to be installed around a spinous process of a vertebra.

2. The anchorage component of claim 1, further comprising a superior connection assembly between the first lateral half and the second lateral half wherein the superior connection assembly is configured to be located above a spinous process when the anchorage component is installed around the spinous process.

3. The anchorage component of claim 2, further comprising an inferior connection assembly between the first lateral half and the second lateral half wherein the inferior connection assembly is configured to be located below a spinous process when the anchorage component is installed around the spinous process.

4. The anchorage component of claim 1, wherein the first lateral half includes a superior end and an inferior end and the first spinous process engagement window is established between the superior end and the inferior end.

5. The anchorage component of claim 2, wherein the first lateral half further comprises a first cutting edge extending from the superior end into the first spinous process engagement window.

6. The anchorage component of claim 5, wherein the first lateral half further comprises an infra laminar hook extending from the inferior end into the first spinous process engagement window.

7. The anchorage component of claim 6, wherein the infra laminar hook extends under a caudal end of laminar when the anchorage component is installed around a spinous process.

8. The anchorage component of claim 1, wherein the second lateral half includes a superior end and an inferior end and the second spinous process engagement window is established between the superior end and the inferior end.

9. The anchorage component of claim 8, wherein the second lateral half further comprises a second cutting edge extending from the superior end into the second spinous process engagement window.

10. The anchorage component of claim 1, wherein the first lateral half further comprises a first spinous process engagement structure extending from the first lateral half adjacent to the first spinous process engagement window.

11. The anchorage component of claim 10, wherein the first spinous process engagement structure is curved to approximate a shape of a spinous process.

12. The anchorage component of claim 11, wherein the first spinous process engagement structure is at least partially flexible.

13. The anchorage component of claim 10, further comprising a protrusion extending from an interior surface of the first spinous process engagement structure.

14. The anchorage component of claim 10, further comprising a first pedicle structure extending from the first spinous process engagement structure.

15. The anchorage component of claim 14, wherein the first pedicle structure is configured to engage an isthmus of the vertebra.

16. The anchorage component of claim 1, wherein the second lateral half further comprises a second spinous process engagement structure extending from the first lateral half adjacent to the first spinous process engagement window.

17. The anchorage component of claim 16, wherein the second spinous process engagement structure is curved to approximate a shape of a spinous process.

18. The anchorage component of claim 17, wherein the second spinous process engagement structure is at least partially flexible.

19. The anchorage component of claim 16, further comprising a protrusion extending from an interior surface of the second spinous process engagement structure.

20. The anchorage component of claim 16, further comprising a second pedicle structure extending from the second spinous process engagement structure.

21. The anchorage component of claim 20, wherein the second pedicle structure is configured to engage an isthmus of the vertebra.

22. A spinal stabilization system, comprising:
a first anchorage component, having a first lateral half and second lateral half, wherein the first lateral half and the second lateral half of the first anchorage component are configured to fit around a spinous process;
a second anchorage component, having a first lateral half and second lateral half, wherein the first lateral half and the second lateral half of the second anchorage component are configured to fit around a spinous process; and
a first longitudinal member configured to be installed at least partially within the first anchorage component and the second anchorage component.

23. The spinal stabilization system of claim 22, wherein the first lateral half of the first anchorage component comprises a first slot and a first setscrew hole adjacent to the first slot and the first lateral half of the second anchorage component comprises a first slot and a first setscrew hole adjacent to the first slot and wherein the first longitudinal member is configured to be installed within each first slot and held in place by a setscrew installed in each setscrew hole.

24. The spinal stabilization system of claim 23, further comprising a second longitudinal member configured to be installed at least partially within the first anchorage component and the second anchorage component.

25. The spinal stabilization system of claim 24, wherein the second lateral half of the first anchorage component comprises a second slot and a second setscrew hole adjacent to the second slot and the second lateral half of the second anchorage component comprises a second slot and a second setscrew hole adjacent to the second slot and wherein the second longitudinal member is configured to be installed within each second slot and held in place by a setscrew installed in each setscrew hole.

26. A method of installing a spinal stabilization system, comprising:
exposing a portion of a spinal column; and
installing a first anchorage component around a first spinous process of the spinal column, wherein the first anchorage component circumscribes the first spinous process.

27. The method of claim 26, further comprising:
installing a second anchorage component around a second spinous process of the spinal column, wherein the second anchorage component circumscribes the second spinous process.

28. The method of claim 27, further comprising:
installing a first longitudinal member between the first anchorage component and the second anchorage component.

29. The method of claim 28, further comprising:
reducing the first longitudinal member to engage each anchorage component.

30. The method of claim 29, further comprising:
installing a first set screw in the first anchorage component to secure the first longitudinal member therein; and
installing a first setscrew in the second anchorage component to secure the first longitudinal member therein.

31. The method of claim 30 further comprising:
installing a second longitudinal member between the first anchorage component and the second anchorage component.

32. The method of claim 31, further comprising:
reducing the second longitudinal member to engage each anchorage component.

33. The method of claim 32, further comprising:
installing a second set screw in the second anchorage component to secure the second longitudinal member therein; and
installing a second setscrew in the second anchorage component to secure the second longitudinal member therein.

34. A kit, comprising:
a plurality of anchorage components, wherein each anchorage component comprises a first lateral half and a second lateral half configured to fit around a spinous process;
a plurality of longitudinal members configured to be installed within each of the plurality of anchorage components; and
a plurality of setscrews configured to bind the longitudinal members within each of the plurality of anchorage components.
